(19) United States
(12) Patent Application Publication
(10) Pub. No.: US 2022/0133128 A1
(43) Pub. Date: May 5, 2022

(54) CREDIT CARD CLEANING DEVICE

(71) Applicant: Justin Tobin Williams, South Jordan, UT (US)

(72) Inventor: Justin Tobin Williams, South Jordan, UT (US)

(21) Appl. No.: 17/518,545

(22) Filed: Nov. 3, 2021

Related U.S. Application Data
(60) Provisional application No. 63/109,678, filed on Nov. 4, 2020.

(51) Int. Cl. A47L 25/00; A61L 2/18

(52) U.S. Cl. CPC A47L 25/00; A61L 2202/17; A61L 2202/121; A61L 2/18

(57) ABSTRACT

A card cleaning device, including a cleaning body, the cleaning body including a rectangular housing, including a first aide and a second side, the second side opposite the first side, a third side and a fourth side, the fourth side opposite the third side, and a fifth side and a sixth side, the sixth side opposite the fifth side, wherein the first side, the third side, and the fifth side are non-coplanar, a slot through the rectangular housing and which only intersects the first and second sides, and a scrubber disposed within the slot, the card cleaning device also including a cover shaped to friction fit about, and cover a majority of the first, second, third, fourth, and fifth sides of the rectangular housing and slidably coupled thereto.

### CROSS-REFERENCE TO RELATED APPLICATIONS

This invention claims priority, under 35 U.S.C. § 120, to the U.S. Provisional Patent Application No. 63/109,678 to Justin Tobin Williams filed on Nov. 4, 2020, which are incorporated by reference berein, in their entirety.

### FIELD OF THE INVENTION

The present invention relates to cleaning devices, and more specifically devices for cleaning plastic cards.

### DESCRIPTION OF THE RELATED ART

Credit cards have become so important in society that the average person cannot always afford to not have one of them constantly on hand. Oftentimes, due to varying circumstances and reasons, most adults carry multiple credit cards around with them at the same time. People carry these credit cards in numerous ways, such as in wallets, purses, bags, dips, and pockets along with their other credit cards and other various items.

Although being incredibly common for the average adult, credit cards are not the only items that people carry around. Many different items are carried not just by adults, but by children as well. Such items include drivers licenses, sporting licenses, insurance cards, identification cards, RFID or other access cards, memberships, and the like are also carried around by people every single day so that they are always able to be accessed and presented when required.

Such cards are also used by people in numerous unkept or otherwise unsanitary places, such as being swiped through, inserted into, or pressed against the registers or access points of many busy stores, gyms, and places of employment, and being banded to a multitude of various merchants, receptionists, or service agents. Oftentimes, these places are used by multitudes of individuals and are rarely cleaned or sanitized on a daily basis, let alone after every user. Even when such places are cleaned, they often have crevices or sensitive electronics such that they are unable to be sanitized as well as desired.

Due to the nature of such places and the amount of use such cards see on a daily basis, the cards quickly become quite dirty and greasy. As such, the cards are very unpleasant to look at or hold, accumulate on them various bacteria and viruses, and transfer all of it. between both the various people and machines used to read the cards, as well as every other place the user puts the card. Such grime compounds as each person's card takes and transmits the grime when in physical contact with another person or machine.

Currently, people who don't want to touch dirty cards resort to using gloves or sanitizing the cards after use, However, cards that are cleaned and sanitized arc generally cleaned and sanitized by hand. Further, this often requires that the user carry around the gloves, or a bottle of sanitizer and a wipe, all of which are awkward to carry and time consuming to use as well as create a lot of waste. This is likely why many cards are simply never cleaned or sanitized at all, leading to the continuous transmittal of grime and bacteria from one spot to another through the card itself,

Some improvements have been made in the field, Examples of references related to the present invention are described below in their own words, and the supporting teachings of each reference are incorporated by reference berein:

U.S. Pat. No. 5,461,747 A. to Clausen et al., discloses a hand-held device for cleaning data-bearing cards includes a housing, a fluid applicator pad, a scrubber pad, a drying pad, and a removable panel. The housing includes a first surface to oppose the portion of the card to be cleaned. A first recess is disposed within the first surface. The first recess has an aperture extending through the housing. A second recess is also disposed adjacent the first recess within the first surface. A third recess is disposed adjacent the second recess within the first surface of the housing. The first, second, and third recesses are generally linearly aligned. The fluid applicator pad is disposed within the first recess. The scrubber pad is disposed within the second recess, The drying pad is disposed within the third recess, Each of the pads is thicker than the depth of its corresponding recess such that the pads project partially above the first surface of the housing. The panel is removably coupled to the housing. The panel includes a bead portion having a second surface oriented generally parallel to the first surface of the housing. The second surface is directly opposed to the first surface for movement of the card therebetween for cleaning.

U.S. Pat. No. 4,104,758 A, to Stotler, discloses a device for washing credit cards comprising an enclosed housing having an inlet aperture through which a credit card holder is insertable for positioning the credit card into a cleaning fluid contained within the housing. Scrubbing brushes submerged in the cleaning fluid within the housing receive the credit card for cleaning the same. The credit card holder is reciprocated with respect to the scrubbing brushes to effect cleaning of the credit card. The housing also functions as a clipboard to hold the charge receipts while the same is being signed by the credit card user.

U.S. Pat. No. 8,770,881 B2, to Dam, discloses sanitization apparatuses, kits, and methods, such as, for example, for pens, styluses, handles, remote controls, and pagers.

U.S. Pat. No. 6,523,600 B1, to Buck et al., discloses a container for wet wipes includes a pop-up style dispensing means formed by a rigid port which surrounds a flexible, rubber-like material or sheet having one or more slits through which the wet wipes are dispensed. The configuration of the slits, in combination with the flexibility of the rubber-like material, enables the user to reach into the container through the slits to grasp a wet wipe in the event the pop-up feature fails, either due to fallback or a missed interfold.

U.S. Patent No. 3,428,988 A, to Dam, discloses a container for cleaning solution that has plural brush bristles between which a card is passed during insertion into and removal from the cleaning solution.

U.S. Patent Application Publication No.. US 2011/014.6012 A1, to Hirst, discloses a sanitizer for disinfecting substantially the entire outer surface of a handheld instrument comprises a container having a well therein for holding a disinfectant and a passageway by which the handheld instrument may be inserted into the well. The sanitizer may include a first opening and a second opening, tire openings being located at opposing ends of the passageway such that the handheld instrument may enter the sanitizer through the fist opening, and exit the sanitizer through the second opening. The sanitizer may further include an absorbent material located in the well to disperse the disinfectant on the outer surface of the handheld instrument, and to prevent the disinfectant from leaking from the well during use.

WIPO Patent Application Publication No.: WO 1999040835 A1, to Bergton, discloses a present invention concerning a cleaning device for information-bearing cards, especially plastic cards with a magnetic strip, data chips or the like. The cleaning device consists essentially of a housing which contains the cleaning element. This housing is provided with bodies which permit the housing to be releasably mounted in a thereto suited holder which in turn can be securely fixed at a location where the cleaning device has to be used. This may be e.g., near a card reader terminal, or in a part of the card reader terminal, e.g. in the extension of the card reader terminal's card track. In a preferred embodiment the housing is provided in its lower portion with bodies which enable the housing to be releasably mounted in a holder placed close to a card reader terminal. This solution permits the actual housing containing the cleaning element for the information-bearing card to easily be replaced by releasing the housing from the fixed or releasably mounted holder and subsequently affixing a new housing with a new cleaning element.

The inventions beretofore known suffer from a number of disadvantages, including but not limited to: drying out quickly, being difficult or requiring specialized tools to replace the sanitizer or other cleaning solution, being complicated, being excessively bulky, being inconvenient to use, using many different parts and pieces, requiring complex assembly, being difficult or time consuming to use, being unreliable or prone to damage, not using a sanitizer or cleaning solution, being difficult to replace sponges or broken components, being increasingly expensive, being difficult to clean or maintenance, taking up large amounts of space, and being difficult to manufacture, being difficult to carry, and/or not being portable.

What is needed is a cleaning device that solves one or more of the problems described berein and/or one or more problems that may come to the attention of one skilled in the art upon becoming familiar with this specification.

### SUMMARY OF THE INVENTION

The present invention has been developed in response to the present state of the art, and in particular, in response to the problems and needs in the art that have not yet been fully solved by the currently available card cleaning devices. Accordingly, the present invention has been designed and developed to provide a satisfactory card cleaning/sanitizing device.

There may be a card cleaning device, which may include a cleaning body, the cleaning body may include one or more of a rectangular housing, which may include a first side and/or a second side, the second side may be opposite the first side, may also or instead include a third side and/or a fourth side, the fourth side may be opposite the third side, and may also include a fifth side and/or a sixth side, the sixth side may be opposite the fifth side, the first side, the third side, and the fifth side may be non-coplanar, the cleaning body may also include a slot which may be through the rectangular housing and may only intersect the first and/or second sides, and a scrubber which may be disposed within the slot, and a cover which may be shaped to friction fit about, and may cover a majority of one or more of the first, second, third, fourth, and/or fifth sides of the rectangular housing and may be slidably coupled thereto, the card cleaning device may include an aperture, the aperture may be through the filth side and may create fluid access to the scrubber, the card cleaning device may include finger slots which may be through the cover, the card cleaning device may include fangs on the cover that grip the grooves on the body, the card cleaning device may include a sixth side which may be substantially flush with an open end of the cover when the cleaning body is fully positioned within the cover, the scrubber may include a sponge which may be trapped to an inner surface of the slot, the rectangular housing may include a first clamshell which may be coupled to a second clamshell, the cover may include a window and/or a stop, either or each may be positioned such that when the rectangular housing is stopped from fully exiting the cover by the stop, the window may provide physical access to the fifth side, and the slot may include a pair of matching clips that may trap the scrubber against an inside surface of the rectangular housing.

Reference throughout this specification to features, advantages, or similar language does not imply that all of the features and advantages that may be realized with the present invention should be or are in any single embodiment of the invention. Rather, the language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments, however that does not mean that those must be present in all embodiments of the invention, rather they may or may not be present in other embodiments.

These features and advantages of the present invention will become more fully apparent from the following description and appended claims or may be learned by the practice of the invention as set forth bereinafter.

### BRIEF DESCRIPTION OF THE DRAWINGS

In order for the advantages of the invention to be readily understood, a more particular description of the invention briefly described above will be rendered by reference to specific embodiments that are illustrated in the appended drawing(s). It is noted that the drawings of the invention are not to scale. The drawings are mere schematics representations, not intended to portray specific parameters of the invention. Understanding that these drawing(s) depict only typical embodiments of the invention and are not, therefore, to be considered to be limiting its scope, the invention will be described and explained with additional specificity and detail through the use of the accompanying drawing(s), in which:

FIG. 1 is a top perspective view of a credit card cleaning device in a closed position, according to one embodiment of the invention;

FIG. 2 is a top perspective view of a credit card cleaning device in an open position, according to one embodiment of the invention;

FIG. 3 is an exploded view of a credit card cleaning device, according to one embodiment of the invention;

FIG. 4 is a side elevational view of a housing of a credit card cleaning device, according to one embodiment of then invention;

FIG. 5 is a side elevational view of a housing of a credit card cleaning device, according to one embodiment of the invention;

FIG. 6 is a side elevational view of a cover of a credit card cleaning device, according to one embodiment of the invention;

### DETAILED DESCRIPTION OF THE INVENTION

For the purposes of promoting an understanding of the principles of the invention, reference will now be made to the exemplary embodiments illustrated in the drawing(s), and specific language will be used to describe the same. It will nevertheless be understood that no limitation of the scope of the invention is thereby intended. Any alterations and further modifications of the inventive features illustrated berein, and any additional applications of the principles of the invention as illustrated berein, which would occur to one skilled in the relevant art and having possession of this disclosure, are to be considered within the scope of the invention.

Reference throughout this specification to an “embodiment,” an “example” or similar language means that a particular feature, structure, characteristic, or combinations thereof described in connection with the embodiment is included in at least one embodiment of the present invention. Thus, appearances of the phrases an “embodiment,” an “example,” and similar language throughout this specification may. but do not necessarily, all refer to the same embodiment, to different embodiments, or to one or more of the figures. Additionally, reference to the wording “embodiment,” “example” or the like, for two or more features, elements, etc. does not mean that the features are necessarily related, dissimilar, the same, etc.

Each statement of an embodiment, or example, is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The features, functions, and the like described berein are considered to be able to be combined in whole or in part one with another as the claims and/or art may direct, either directly or indirectly, implicitly or explicitly.

As used berein, “comprising,” “including,” “containing,” “is,” “are,” “characterized by,” and grammatical equivalents thereof are inclusive or open-ended terms that do not exclude additional unrecited elements or method steps. “Comprising” is to be interpreted as including the more restrictive terms “consisting of” and “consisting essentially of.”

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims or may be learned by the practice of the invention as set forth bereinafter.

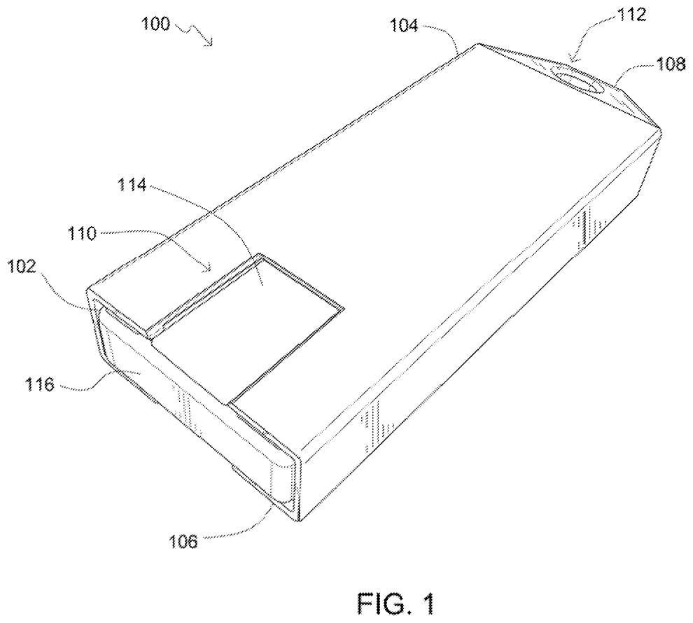

FIG. 1 is a top perspective view of a credit card cleaning device in a closed position, according to one embodiment of the invention. There is shown a credit card cleaning device 100, including a housing 102 and a cover 104. The cover 104 includes a from end 106 and a back end 108 opposite the front end 106, the from end 106 including a window 110 and the back end 108 including an attachment point 112. The front end 106 is shown as being hollow and includes a window 110 extending into the body of the cover 104 from the front end, however in other embodiments the window 110 may be placed elsewhere or there may be no window. The back end 108 is shown as being tapered to provide structural rigidity to the cover 104 as well as more rotational flexibility for the device attached to the attachment point 112.

The illustrated housing 102 includes a visible third side 114 and a sixth side 116. As shown, the credit card cleaning device 100 is in a closed position, wherein the housing 102 is substantially covered by the cover 104. When in the closed position, the housing 102 is fully positioned within the cover 104 and the sixth side 116 is substantially flush with the front end 106 of the cover 104. While in the closed position, the sixth side remains substantially uncovered and the third side 114 remains accessible to the user from the window 110.

In the illustrated embodiment, the housing 102 and cover 104 are connected to form the complete credit card cleaning device 100. When combined, there is a tight fit between the housing 102 and the cover 104, so that the housing 102 and cover 104 do not slide relative to one another without an outside force nor have any gaps between where they touch.

Advantageously, the credit card cleaning device 100 allows a user to easily and conveniently sanitize and clean credit cards and other similarly shaped objects without necessitating a complex, bulky, or otherwise unwieldly device around. Further, the credit card cleaning device doesn't leak and remains damp with cleaning solution or sanitizer for long periods of time, even when exposed to the elements. Therefore, not only can the device be carried with the user at all times, it also can be stored in areas where cleaning or sanitizing is necessary.

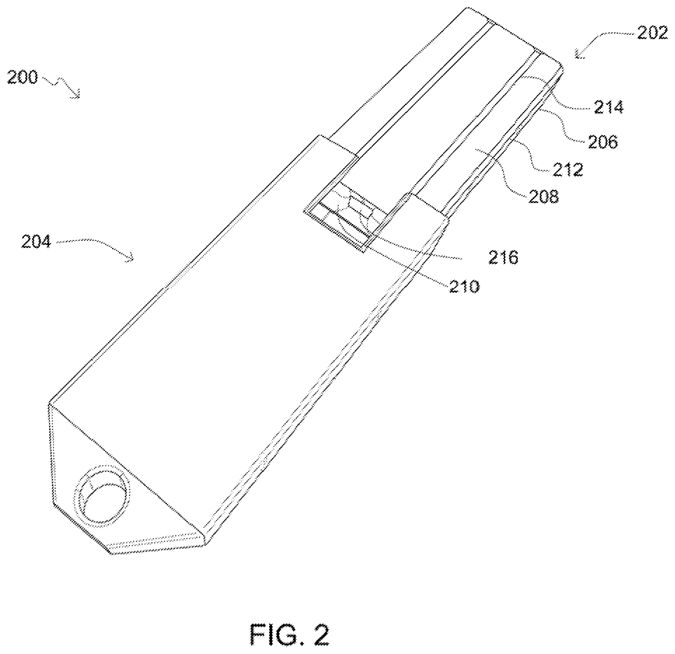

FIG. 2 is a top perspective view of a credit card cleaning device in an open position, according to one embodiment of the invention. There is shown a credit card cleaning device 200, including a housing 202 and a cover 204. As shown, the credit card cleaning device 200 is in an open position, wherein the cover 204 does not substantially cover the housing 202, thereby permitting access to the cleaning structures of the device.

As shown, the housing 202 includes a first side 206, a third side 208, and a fifth side 210. The first side 206 includes a slot 212 which passes through the housing and out the side opposite the first side 206. The slot 212 is formed such that an object such as a credit card may be slid in the slot 212 on one side, through the credit card cleaning device 200, and pulled out the other side of the slot 212. Generally, the distance between the first side 206 and the side opposite the first side 206 slots is small enough where a credit card is able to be grabbed from the opposite side slot before it is fully passed through the first side 206 slot 212, however the device and slots 212 may be designed so that a user can continue to push the card, either with their fingers or another device, through the slot 212 once it is flush with the slot 212.

In a non-limiting embodiment the device contains a plurality of slots for allowing more than one user to use the device or allow for the cleaning of multiple cards at one time. In another non-limiting embodiment, the device contains only one slot where a user inserts a card, pulls it out of the same slot, and reinserts it in the reverse direction to clean the other side of the card (similar to a gas pump card reader or chip reader). In a third non-limiting embodiment, the slot extends the full length of the device and the user swipes the card through the device and then may or may not need to flip the card and swipe again to clean the rest of it (similar to a credit card swiping machine, it is contemplated that the device may be attached to one or both ends of one instead of being on its own as a separate unit).

The third side 208 is shown to include grooves 214 which line up with protrusions within the cover 204 which keeps the housing 202 and cover 204 in line and from becoming pinched with respect to one another. The grooves 214 also keep the housing 202 from pulling all the way out of the cover 204 as the grooves 214 do not extend all the way along the third side 208 which creates a stop which catches the protrusions of the cover 204.

In a non-limiting embodiment, the grooves 214 may allow the housing 202 to fully extend out of the cover 204 such that the two sections are no longer a single unit. In another non-limiting embodiment, the grooves 214 may have indents such that the housing 202 may be pulled out in increments and remains at certain intervals when released. In other non-limiting embodiments, there may be no grooves, a single groove, or a plurality of grooves.

The fifth side 210 includes an aperture 216 which extends into the body of the housing 202. In the illustrated embodiment, the aperture 216 meets with the slot 212 such that access to the skit 212 can be had via the aperture 216. When in the open position, the fifth side 210 is exposed through a window in the cover 204 such that physical access to the aperture 216 is possible (e.g. for refilling the device with cleaning fluid).

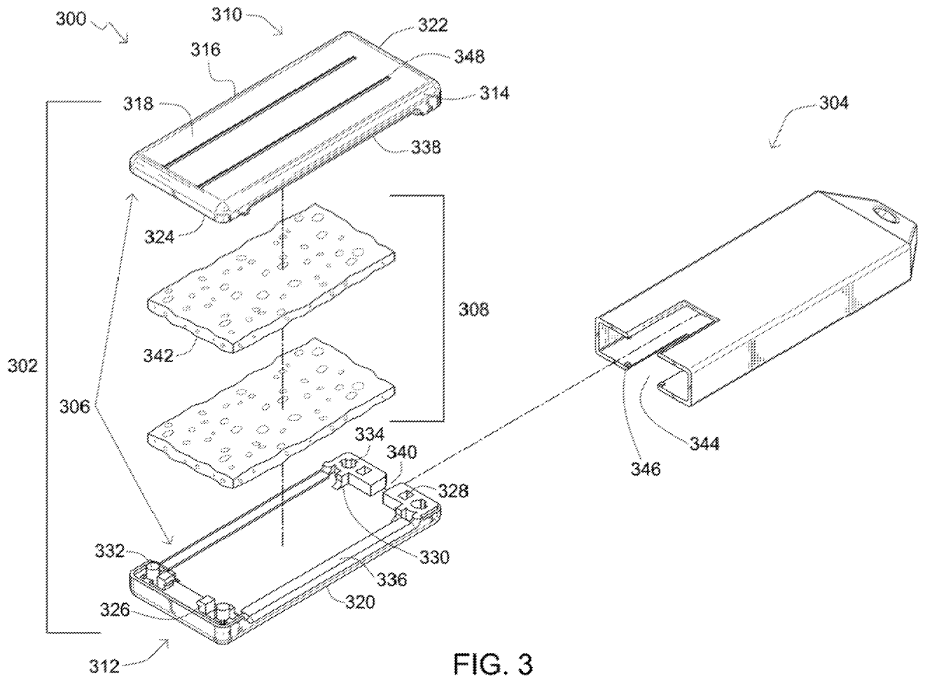

FIG. 3 is an exploded view of a credit card cleaning device, according to one embodiment of the invention. There is shown a credit card cleaning device 300 including a cleaning body 302 and a cover 304. The illustrated cleaning body 302 includes a pair of cover sections (top and bottom 306) that sandwich a pair of scrubbers 308 therebetween such that when a card is inserted into an aperture formed by inset edges 338 of the cover sections 306 the scrubbers 308 clean the inserted card. Of note, there are no complicated mechanical parts, no electrical parts, no seals and the like that may fail or wear out. The illustrated embodiment is a simple cost-effective embodiment such that the device can be easily repaired or portions replaced, however in other embodiments the device may be more or less sections.

The illustrated cleaning body 302 includes a housing 306 and a scrubber 308. As shown, the housing includes a top section 310 and a bottom section 312. The top section 310 includes a first side 314, a second side 316, a third side 318, a fifth side 322, and a sixth side 324. The bottom section 312 includes a first side 314, a second side 316, a fourth side 320, a fifth side 322, and a sixth side 324. The first side 314 and second side 316 of the top section 310 and bottom section 312 are shown opposite one another, as are the fifth side 322 and sixth side 324 of the top section 310 and bottom section 312. When combined, the third side 318 of the first section 310 and the fourth side 320 of the bottom section 312 are opposite one another. As shown, the first side 314, third side 318, and fifth side 322 and their respective opposite side are not coplanar with any of the other sides.

The illustrated top section 310 and bottom section 312 each include a rectangular body with filleted edges, however in other embodiments may be any number of shapes with or without filleted or chamfered edges. The top section 310 and bottom section 312 are shown to each further contain two clasps 326 and two clasp seats 328 as well as four supports 330 to assist in bolstering the strength of the top section 310 and bottom section 312, as well as holding objects securely within the credit card cleaning device 300. The top section 310 and bottom section 312 also contain two pegs 332 and two peg seats 334, the seats 334 opposite the pegs 332. As illustrated, the top section 310 and bottom section 312 further include a pair of matching clips 336 that trap the scrubber 308 against an inside surface of the housing 306. There matching clips run parallel to one another along the side of the top section 310 and bottom section 312.

As shown, the top section 310 and bottom section 312 of the housing 306 are each half of a clamshell, such that the clamshells of the top section 310 and bottom section 312 fit together to form the complete housing 306. As assembled in the illustrated embodiment, inset edges 338 on the top section 310 and bottom section 312 line up. When pressed together, the top section 310 and bottom section 312 further define a slot. The slot is shown to intersect the top section 310 and bottom section 312 and extends through each. The illustration shows that the slot only intersects the first side 314 and second side 316 of the top section 310 and bottom section 312. The top section 310 and bottom section 312 are shown hollowed out in order to reduce the total weight and required material of the credit card cleaning device 300.

Further, the illustrated embodiment shows that the top section 310 and bottom section 312, when pressed together, also define an aperture 340. As shown, the aperture 338 is located through the side 322 of the top section 310 and bottom section 312 and intersects the interior area defined by the slot. Accordingly, the aperture 340 in turn creates a fluid access point in the housing 306 for the scrubber 308. The top section 310 and bottom section 312 are thick enough to provide a desired strength and heft without being overly bulky.

The hollowed-out portion of the top section is designed to hold a scrubber 308. The illustrated scrubber 308 is disposed within the interim area defined by the slot, and the scrubber 308 further includes a sponge 342 which ma be trapped against or to an inner surface of the top section 310 and/or the bottom section 312. The scrubber 308 may be any number of items, materials or combinations thereof, such as a foam pad, sponge, cloth, array of bristles, woven fibers, or other object that can absorb or otherwise carry hand sanitizer, soap, or other cleaning solution and provide abrasion to a card as it passes alongside thereto. Generally, the scrubber 308 will be able to both sanitize and clean the device being passed through the credit card cleaning device 300.

The scrubber 308 may be replaceable or refillable, and in a non limiting embodiment there may be one or more reservoirs to assist in feeding the scrubber 308 over time, refilling the scrubber 308, and/or providing a hand sanitizing dispenser for the user to use for their own hands. Additionally, there may be more than one scrubber 308 stored within the device, such as a top and bottom scrubber or multiple different scrubbers that perform different tasks, such as a coarse or rough scrubbing device or bristles to assist in cleaning dirt and grime and/or a soft and absorbent scrubber to dispense sanitizer, soap, or other cleaning solutions onto the card in order to clean it of bacteria, viruses, and/or grease and slime.

The illustrated cover 304 is shaped to friction fit about, and cover a majority of the first side 314, second side 316, third side 318, fourth side 320, and fifth side 322 of the housing 306 and slidably coupled thereto. The cover 304 further includes a window 344, which as illustrated expose a section of the third side 318 and the fourth side 320 of the housing 306 when the credit card cleaning device 300 is in a closed position. Accordingly, a user is able to grasp the housing 304 even when the device is in the closed position.

Further the third side 318 and fourth side 320 include grooves which extend along a majority of the third side 318 and fourth side 320 of the housing 304. The cover 304 also includes fangs 346 that grip the grooves 342 on the housing 304 and help guide and restricts the housing 304 as it moves between the open and close positions and is positioned within the cover 304. The cover 304 further includes a fast stop 348, which is positioned such that when the housing 306 is stopped from fully exiting the cover when the fangs 346 contact the first stop 348.

In other non-limiting embodiments, attached to the top section 310 and/or bottom section 312 is a clip and attached to the side opposite the clip is a mount, however these clips and mounts are not limited to only being opposite one another in designs. One or more of the clips may be substituted for a hinge which connects to the top section and/or bottom section and may be formed with either the top or bottom section. This would allow the top and bottom sections to stay connected when inserting, removing, or changing the cleaning and/or sanitizing device. Additionally, although there is an attachment point attached to the cover, a keyring hole, bracket, or other mounting device may be integrated into the top and/or bottom sections or attached to the outside of the device to aid a user in securely installing the device in a desired location or the ability to attach it to something to secure it or carry it with them.

The top section 310 and bottom section 312 are generally made completely each as one complete piece; however, it may be that creating one or more pieces separately and attaching them later is more suitable. The credit card cleaning device 300 is generally made completely of a strong, lightweight, and durable material such as a hard plastic, metal, carbon fiber, or ceramic, however it is envisioned that softer plastics and other materials may be beneficial for specific uses, as well as combinations of different materials.

Although not explicitly stated, the illustrated bottom section 312 is substantially identical to the top section, and when turned over and rotated, structure on the bottom section 312 lines up with the corresponding structure on the top section 310, and vice versa, This means that the clasps 326 the pegs 332 on the bottom section 312 should line up with the clasp seats 328 and peg seats 334 on the top section 310, and vice versa. In other non-limiting embodiments, the top side 310 and bottom side 312 are not substantially identical pieces and differ in one or more ways, such as but not limited to: shape, size, number of pieces, types of components, or are exactly identical.

The top section 310 and/or bottom section 312 may also be covered, coated, or plated with a material or combination of materials to make the credit card cleaning device better by: increase strength, help weatherproof, create a solid seal, increase friction, increase impact resistance, sanitize or remain sanitary, or others, Coatings, coverings, or plating may include but are not limited to the following materials: metals, hard or soft plastics, silicones, adhesives, grip coatings, rubbers, carbon fiber, sanitary compounds or sprays, or combinations of materials.

In one non-limiting embodiment, there is a credit card cleaning device that can be integrated into or otherwise attached to another device, such as a gas station pump (one non-limiting embodiment has the unit placed in front of the card reader so that the card is cleaned and sanitized as the user normally inserts the card), retail counter, or other device/structure. It is also portable so that it may be personally used, stored, and carried. It may be an “anywhere” credit card cleaning device. There may be multiple versions and/or variations, one embodiment being an industrial version which is designed to be mounted and remain durable in the intended situation, long lasting or easy to refill, and used often by many people. Another embodiment may be a personal version which is portable, able to be transported without leaking, and intended to be used somewhat infrequently or even disposable.

To assemble the illustrated embodiment, a user would first insert one or more scrubbers 308 within the hollowed-out portion of the top section 310 and/or bottom section 312 defined by the slot. Afterwards, a user would simply need to line up the pegs 332 and clasps 326 on the top section 310 with the peg seats 334 and clasp seats 328 on the bottom section 312, and vice versa. The user would then push the fifth side 322 of top section 310 and bottom section 312 towards one another, until the clasps 326 snap into place, and would then push the sixth side 324 of top section 310 and bottom section 312 towards one another until the clasps 326 snap into place. This may be done vice versa, and a skilled user may be able to do both the fifth side 322 and the sixth side 324 at the same time.

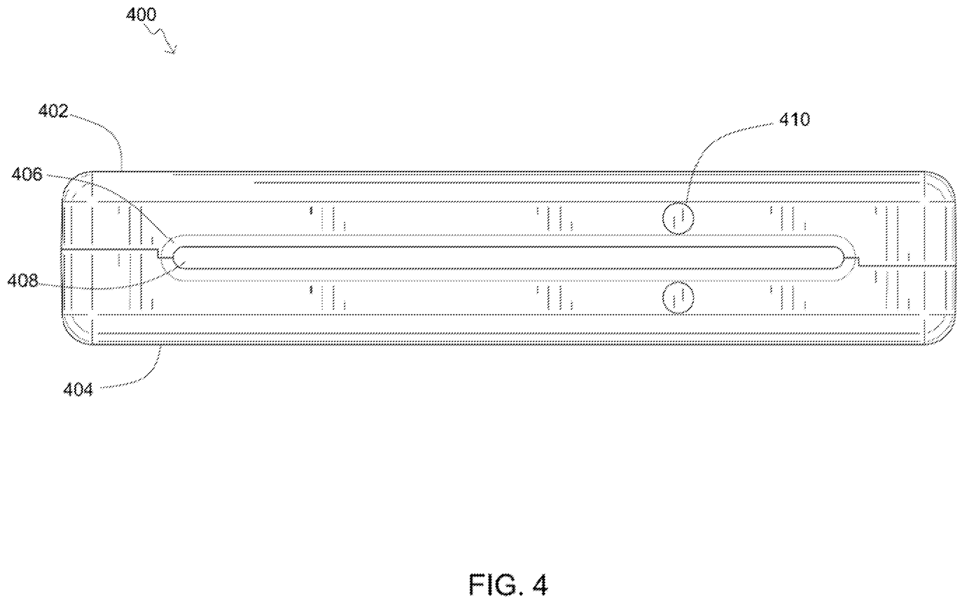

FIG. 4 is a side devotional of a housing of a credit card cleaning device, according to one embodiment of then invention. In the illustrated embodiment, the housing 400 is assembled such that the top section 402 is mated to the bottom section 404. As can be seen, when mated, there are inset edges 406 on the top section 402 and bottom section 404 which line up to form a slot 408. Further, the inset edges 406 are filleted such that a card is able to easily slide into the slot 408 even if the user slightly misses the slot 408. Further, there are two indents 410 which match with protrusions on the cover such that the housing 402 stays within the cover when in the closed position, and is only removable when an outside force is applied.

In other non-limiting embodiments, the dimensions of the slot 408 may be different, as well as the entire orientation of the slot 408 may be different, or even the general shape changed of the slot may be changed. Additionally, the top section 402 and bottom section 404 may be secured to one another with by other various means, including but not limited to: adhesives, screws, bolts, clamps, hinges, clips, and/or formed together with a flexible plastic or rubber. The device may also be formed as one piece which is disposable once used up, a cleaning device that does not get replaced and is refilled only or involves pulling out the cleaning device from a slot or other compartment in order to change it.

In a non-limiting embodiment, the slot may also contain one or more seals, including but not limited to flexible seals or gasket, flaps, or a slidable or hinged door in order to keep air, dirt, and other contaminants out of the device as well as keeping whatever sanitizing and cleaning object that is inside the device from leaking, drying out, or being exposed to the environment.

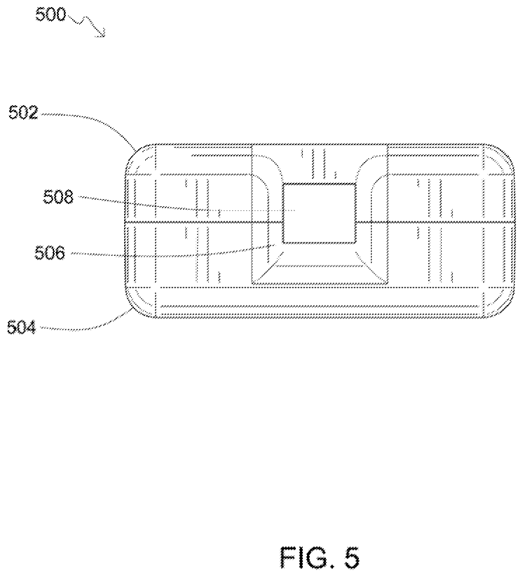

FIG. 5 is a side elevational view of a housing of a credit card cleaning device, according to one embodiment of the invention. In the illustrated embodiment, the housing 500 is assembled such that the top section 502 is mated to the bottom section 504. As can be seen, when mated, there are inset edges 506 on the top section 502 and bottom section 504 which line up to form an aperture 508. The aperture 508 is formed such that replacement sanitizer and/or cleaner may be placed into the housing 500. Further, the inset edges 506 are filleted such that the sanitizer or cleaner runs or drips info the aperture 508 even if the user slightly misses the aperture 508. Further, the aperture 508 is shown to be asymmetrical; however in other embodiments, the aperture 508 may be symmetrical with respect to both the top section 502 and the bottom section 504, and/or may be shifted from the center of the device.

In other non-limiting embodiments, the dimensions of the aperture 508 may be different, as well as the entire orientation of the aperture 508 may be different, or even the general shape changed of the aperture 508 may be changed. In a non-limiting embodiment, the aperture 508 may also contain one or more seals, including but not limited to flexible seals or gasket, flaps, or a slidable or hinged door in order to keep air. dirt, and other contaminants out of the device as well as keeping whatever sanitizing and cleaning object that is inside the device from leaking, drying out, or being exposed to the environment.

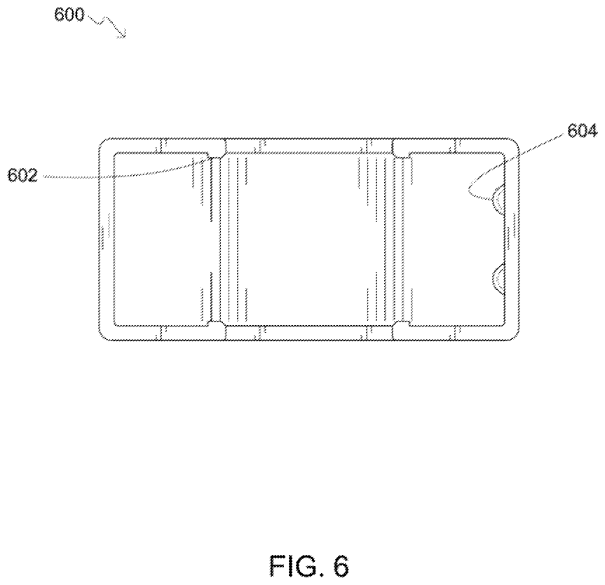

FIG. 6 is a side elevational view of a cover of a credit card cleaning device, according to one embodiment of the invention. As illustrated the cover 600 includes a plurality of fangs 602 which protrude from the interior of the housing 600 and are designed to secure the cover 600 to the housing of the credit card cleaning device. In other embodiments, there may be no fangs, a single fang, or a plurality of fangs, and/or the fangs may be any number of shapes and sizes.

As illustrated, the cover 600 further includes a plurality of protrusions 604 within the cover 600 which mate with indentations on the housing. When mated, the protrusions 604 and indentations hold the cover 600 to the housing and require an outside force to remove the housing from being fully positioned within the cover 600. In other embodiments, there may be no protrusions, a single protrusion, or a plurality of protrusions, and/or the protrusions may be any number of shapes and sizes to mate with the indents.

It is understood that the above-described embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claims rather than by the foregoing description. All changes which come within the meaning and range of equivalency of the claims are to be embraced within their scope.

Further, while embodiments having two sections each containing two pegs, two ribs, four supports, one clip, and one mount have been shown, fewer, additional, or entirely different sections may be coupled together, and each section may have fewer or additional components.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims. Further, it is contemplated that an embodiment may be limited to consist of or to consist essentially of one or more of the features, functions, structures, methods described herein.

What is claimed:

1. A card cleaning device, comprising:
a. a cleaning body, including:
i. a rectangular housing, including a first side and a second side, the second side opposite the first side, a third side and a fourth side, the fourth side opposite the third side, and a fifth side and a sixth side, the sixth side opposite the fifth side, wherein the first side, the third side, and the fifth side are non-coplanar;
ii. a slot through the rectangular housing and which only intersects the first and second sides; and
iii. a scrubber disposed within the slot; and
b. a cover shaped to friction fit about, and cover a majority of the first, second, third, fourth, and fifth sides of the rectangular housing and slidably coupled thereto;

2. The device of claim 1, wherein the housing further includes an aperture, the aperture located through the fifth side and creating fluid access to the scrubber.

3. The device of claim 1, wherein the cover further includes finger slots.

4. The device of claim 1, wherein the housing further includes grooves on the third and fourth sides, and the cover further includes fangs that grip the grooves on the housing.

5. The device of claim 1, wherein the sixth side is substantially flush with an open end of the cover when the cleaning body is fully positioned within the cover.

6. The device of claim 1, wherein the scrubber further includes a sponge trapped to an inner surface of the slot.

7. The device of claim 1, wherein the rectangular housing further includes a first clamshell coupled to a second clamshell.

8. The device of claim 1, wherein the cover further includes a window and a first stop, each positioned such that when the rectangular housing is stopped from fully exiting the cover by the first stop, the window provides physical access to the fifth side.

9. The device of claim 1, wherein the slot further includes a pair of matching clips that trap the scrubber against an inside surface of the rectangular housing.

10. The device of claim 1, wherein the cover further includes a second stop which requires an outside force to remove the cleaning body from being fully positioned within the cover.

11. A card cleaning device, comprising:
a. a cleaning body, including:
i. a rectangular housing, including a first side and a second side, the second side opposite the first side, a third side and a fourth side, the fourth side opposite the third side, and a fifth side and a sixth side, the sixth side opposite the fifth side, wherein the first side, the third side, and the fifth side are non-coplanar;
ii. a slot through the rectangular housing and which only intersects the first and second sides; and
iii. a scrubber clipped to an inside surface of the slot; and
b. a cover shaped to friction fit about, and cover a majority of the first, second, and fifth sides of the rectangular housing and slidably coupled thereto:

12. The device of claim 11, wherein the housing farther includes an aperture, the aperture located through the fifth side and creating fluid access to the scrubber.

13. The device of claim 12, wherein the cover further includes finger slots.

14. The device of claim 13, wherein the housing further includes a groove and the cover further includes a fang that grips the groove on the housing.

15. The device of claim 14, wherein the sixth side is substantially flush with an open end of the cover when the cleaning body is fully positioned within the cover.

16. The device of claim 15, wherein the scrubber includes a sponge trapped to an inner surface of the slot.

17. The device of claim 16, wherein the rectangular housing includes a first clamshell coupled to a second clamshell.

18. The device of claim 17, wherein the cover includes a window and a stop, each positioned such that when the rectangular housing is stopped from fully exiting the cover by the stop, the window provides physical access to the fifth side.

19. The device of claim 18, wherein the cover further includes a second stop which requires an outside three to remove the cleaning body from being fully positioned within the cover.

20. A card cleaning device, comprising:
a. a cleaning body, including:
i. a rectangular housing, including a first side and a second side, the second side opposite the first side, a third side and a fourth side, the fourth side opposite the third side, and a fifth side and a sixth side, the sixth side opposite the fifth side, wherein the first side, the third side, and the fifth side are non-coplanar, and wherein the third and fourth sides include a plurality of grooves;
ii. a slot through the rectangular housing and which only intersects the first and second sides;
iii. a scrubber clipped to an inside surface of the slot; and
iv. an aperture, the aperture located through the fifth side and creating fluid access to the scrubber; and
b. a cover shaped to friction fit about, and cover a majority of the first, second, and fifth sides of the rectangular housing and slidably coupled thereto, wherein the cover includes linger slots and further includes a plurality of fangs that grip the grooves on the housing;
